(19) United States
(12) Patent Application Publication
(10) Pub. No.: US 2023/0233395 A1
(43) Pub. Date: Jul. 27, 2023

(54) DEVICE FOR APPLYING A TEMPERATURE EFFECT TO THE HUMAN BODY

(71) Applicant: Ilya Viktorovich Osipov, Nizhny Novgorod (RU)

(72) Inventor: Ilya Viktorovich Osipov, Nizhny Novgorod (RU)

(21) Appl. No.: 18/007,748

(22) PCT Filed: Jan. 1, 2021
(86) PCT No.: PCT/RU2021/050001

§ 371 Date: Dec. 2, 2022

(30) Foreign Application Priority Data
Jun. 2, 2020 (RU) 2020118184

(51) Int. Cl. A61H 1/00

(52) U.S. Cl. CPC A61H 1/00; A61H 2201/0207; A61H 2201/0285; A61H 2201/5097

(57) ABSTRACT

The proposed device for a thermal treatment of a human user includes contact elements (plates) for contacts with a user's body connected to thermal sources electrically coupled to a power module and to a control module configured for thermal massage of the user's body. The thermal elements are located on the common base. Each element includes a thermo-conductive plate connected to a Peltier element. The elements have apertures for outward air flow and air fans connected to the power module and to the controller module. The elements' air fans are positioned in such a way that the air is directed through the thermos-conductive radiators in the direction to the apertures. A thermo-conductive plate of each of the thermal elements is connected to the thermos-conductive radiator. The Peltier elements are connected to the power module and to the controller module.

### FIELD OF INVENTION

This disclosure is related to devices for thermal treatment of a human user including contact elements with a body if the user connected with thermal sources electrically connected to a power module and to a control module configured for thermal massage of the user's body.

### BACKGROUND

Massage positively affects people. The massage can be tactile and/or thermal such as, for example, contrast shower (cold and hot alternation). In order to provide for efficient contrast shower, devises affecting separate part of user's body that have many nerve endings are used.

For example, a device for thermal influence on a human that uses contact plates for touching the user's body connected to the thermal elements electrically coupled with a power module and a control block is disclosed in Patent RU No 2134096, issued in 1999.

This device for thermal influence of a human is the closes in terms of technical essence and by the achieved effects and is selected as a prototype of the current invention.

A shortcoming of this prototype is low effectiveness of the device. The prototype device does not provide for fast switching of thermal regimes from cooling to heating and vice versa.

### DETAILED DESCRIPTION

The proposed devices for a thermal treatment of a human user includes body contact elements (plates) connected to thermal sources electrically coupled to a power module and to a control module configured for thermal massage of the user's body. The proposed device solves the low efficiency issue of the known massage devices.

According to the disclosed embodiment, the thermal elements are located on the common base. Each element includes a thermo-conductive plate connected to a Peltier element. The elements have apertures for outward air flow and air fans connected to the power module and to the controller module. The elements' air fans are positioned in such a way that the air is directed through the thermos-conductive radiators in the direction to the apertures. A thermos-conductive plate of each of the thermal elements is connected to the thermos-conductive radiator. The Peltier elements are connected to the power module and to the controller module.

Accordingly, the disclosed device for the thermal treatment provide for a rapid change of temperature of each affected area from cold to hot and vice versa thereby facilitating various patterns of thermal treatment based on movement of thermal waves in different directions. The disclosed device provides for increased speed of changing surface temperature by fast cooling provided by extracting the heat through the apertures using the air fans.

In one embodiment, the controller may be connected to a remote-control device. This provides for remote controlling of heating and cooling parameters. In one embodiment, the controller may have control buttons located on the outer surface of its housing. This allows for additional control of heating and cooling parameters.

In one embodiment, the base of the proposed device is made of a material of various plasticity that also facilitates control of heating and cooling parameters.

In one embodiment, the controller may be configured to be connected to a wireless network for connections to external devices.

The disclosed thermal treatment device is flexible for contacting any areas of the user and yet it can be configured for a particular body part and morph around that part for maximum contact for a maxim treatment effect.

### BRIEF DESCRIPTION OF DRAWINGS

A more particular description of the present disclosure will be rendered by reference to specific embodiments thereof that are illustrated in the appended drawings.

It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 reflects a schematic view of the device for a thermal treatment of a human, according to the invention;

FIG. 2 reflects construction of a separate element of the device for a thermal treatment of a human, according to the invention;

FIGS. 3 and 4 depict an external view of the device for a thermal treatment of a human, according to the invention;

FIGS. 5 and 6 depict an external view of a disposition of elements within the device for a thermal treatment of a human, according to the invention.

The figures depict the following:

1—an element of a thermal application,

11—thermo-conductive plate,

12—Peltier elements,

13—thermo-emitting radiator,

14—a base layer for a common flexible positioning of elements,

2—a power module,

3—a controller block,

4—common flexible base,

5—outlet aperture,

6—air fan.

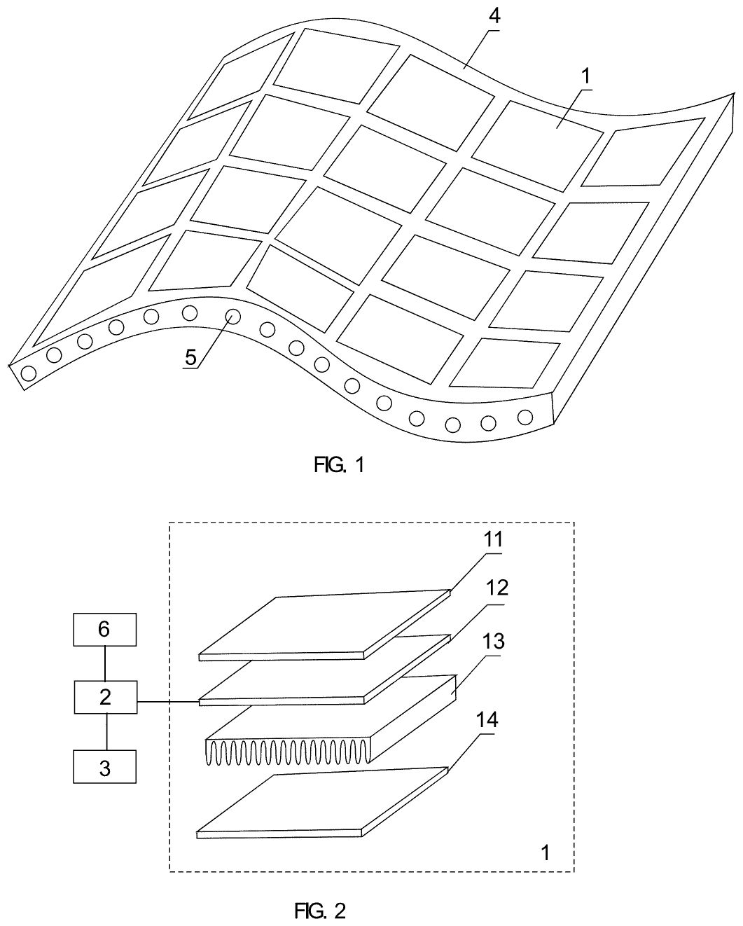

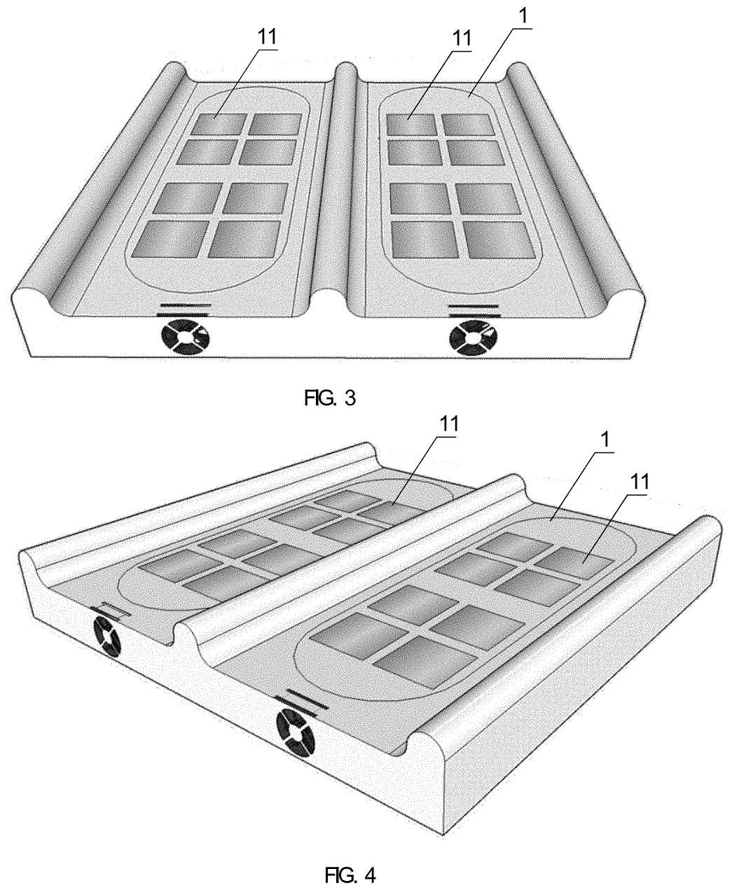

According to FIGS. 1 and 2, the device for a thermal treatment of a human includes contact plates configured to be in contact with user's body. The contact plates are connected to the thermal elements electrically coupled with the power module 2 and with the controller block 3. The device for a thermal treatment of a human is implemented as several separate thermal elements 1 located on a common flexible base 4. Each thermal element includes a thermo-conductive plate 11 connected to the Peltier element(s) 12, which is in turn connected to the thermo-emitting radiator 13. The Peltier element(s) 12 of each thermal element are connected to the power module 2 and to the controller block 3.

Each thermal element 1 may have a base layer 14 configured for positioning of the element on the common flexible base 4.

Some thermal elements 1 may have outlet apertures 5 for passing the air out of the thermal elements using the air fans 6 located inside the thermal elements 1 and connected to the power module 2 and to the controller block 3. The air fans 6 may be positioned in such a way that the air is directed through the thermo-emitting radiators 13 to the outlet apertures 5.

The controller block 3 may be connected to the external remote-control device. The controller block 3 may have a wireless network module for connecting to the external devices.

As discussed above, the flexible base may have various flexibility and may be made from a non-flexible material such a hard plastic or from a soft material. The base layer may be flexible (slightly soft) for a better contact with a user's body.

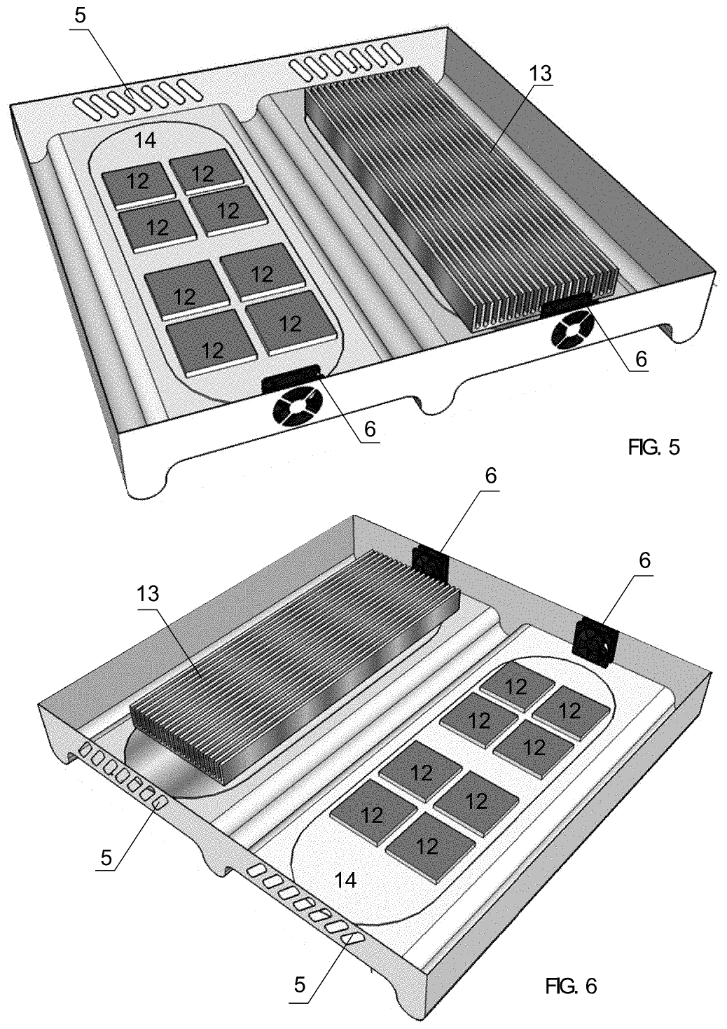

The thermo-emitting radiator 13 in FIGS. 5 and 6 may be implemented as a common part or it may be divided into sections.

### Implementation of the Invention

The device for a thermal treatment of a human operates as follows. This implementation is a non-limiting example.

Stage 1. The device for a thermal treatment is placed on a body of a user, for example on an arm or on a leg.

Stage 2. Select an operation mode for the thermal treatment of the user via the controller block.

Stage 3. Send a control signal to each of the Peltier elements 12 to either quickly heat up or cool down the element based on the selected mode of operation.

Stage 4. A thermo-conductive plate 11 connected to the Peltier elements 12 passes the heat or the cold to the body of the user thereby providing a thermal massage.

Stage 5. If the thermo-conductive plate 11 needs to be cooled off fast, the air fans 6 force the air through the thermo-emitting radiators 13 to the outlet apertures 5.

### Commercial Applicability

The proposed device for a thermal treatment of a human may be easily used by a partitioner that meets the criteria of a “commercial applicability.”

A prototype of the device for the thermal treatment of a human had been made. The tests of the device for the thermal treatment of a human showed that the device provides for high effectiveness due to ability to switch a thermal operation mode from heat to cool. Also, the proposed device allows for thermal application in opposite phases—i.e., when one portion of the user's body is being heated while another portion is being cooled off and vice versa. These features produce the desired technical result of improved effectiveness of the thermo-massaging device.

1. A device for a thermal treatment of a human, the device comprising:
a plurality of contact plates configured to come in contact with a user's body and connected to thermal elements wherein the thermal elements are electrically connected to a power module and to a controller block,
wherein the thermal elements are located on a common base and each of the thermal elements comprises a thermo-conductive plate connected to a Peltier element,
the thermal elements comprise outlet apertures and air fans connected to the power module and to the controller block, wherein the thermal elements are positioned to direct air flow through thermo-emitting radiators to the outlet apertures, and
wherein the thermo-conductive plate of each of the thermal elements is connected to the thermo-emitting radiator, and the Peltier element is connected to the power module and to the controller block.

2. The device of claim 1, wherein the controller block is connected to an external remote-control device.

3. The device of claim 1, wherein the controller block comprises a wireless module for connection to external devices.
